(19) United States
(12) Patent Application Publication
(10) Pub. No.: US 2022/0081671 A1
(43) Pub. Date: Mar. 17, 2022

(54) In-Process Device and Method for Cell Culture Monitoring

(71) Applicant: Global Life Sciences Solutions USA LLC, Marlborough, MA (US)

(72) Inventors: Swapnil Puranik, Bengaluru, Karnataka (IN); Manoj Kumar Ramakrishna, Bengaluru, Karnataka (IN); Haresh Patil, Bengaluru, Karnataka (IN)

(21) Appl. No.: 17/413,721

(22) PCT Filed: Dec. 18, 2019
(86) PCT No.: PCT/EP2019/086064

§ 371 Date: Jun. 14, 2021

(30) Foreign Application Priority Data
Dec. 21, 2018 (IN) 201841048612

(51) Int. Cl. C12M 1/34; G06T 7/00; G06T 7/20; C12M 3/06

(52) U.S. Cl. CPC C12M 41/46; G06T 7/0012; G06T 7/20; G06T 2207/30242; C12M 41/44; G06T 2207/10056; G06T 2207/30024; C12M 27/16

(57) ABSTRACT

Disclosed is an in-process cell monitoring device comprising: a flow channel having at least one inlet and at least one outlet exposeable to a cell culture; a microscope positionable to view the contents of a region of the channel; and a computer operable at least to count any cells in the region, providing a closed fluid circuit for cell monitoring. Disclosed also is a bioreactor including a cell culture volume, and an in-process cell monitoring device, said device comprising: a flow channel having at least one inlet and at least one outlet each in fluid communication with the volume, of sufficient cross-sectional area to allow fluids to drain from the inlet to the outlet; and a microscope positionable to view the contents of a region of the channel, and also A method for monitoring a cell culture including determining cell density.

### TECHNICAL FIELD

The present invention relates to a device for in-process monitoring of cell density and cell viability by means of cell imaging, for example in the field of immunotherapeutic cell cultivation.

### BACKGROUND

The important parameters for cell growth, which usually mimic their in vivo state, are generally well recognised presently and for specific cells lines, for example those used to express monoclonal antibodies, their cell culture parameters can be predicted reasonably accurately, based on previous experience. Thus, cell culturing, even with continuously fed batch culturing, can be readily optimised. However, where the characteristics of seed cells of a cell culture batch, for example in single batch autologous immunotherapeutic cell culture, are largely unknown, or highly variable, then to some degree, the cell culture parameters have to be adjusted based on culture sampling during the culturing process, in order to optimise that process.

Various sensors are available commercially to detect in-process cell culture parameters, for example, to detect concentrations of CO2, O2 and N2 gases, and temperature, and these can be used to provide known satisfactory culture conditions. However, for unknown cell characteristics, it is the resultant cell activity that is important, rather than the exact input parameters, so it is more important to monitor the effectiveness of the input parameters. Known measures of effective cell culture conditions are: cell density-cell numbers in a defined area or volume of fluids (over time providing an indication of cell division rates); cell viability—e.g. the number of healthy cells observed in a defined area or volume of fluid; metabolites and nutrients, for example glucose, lactate, and ammonium concentrations; expression of certain proteins (for example to determine the ratio of killer and helper T cells and determine functionally active cells); and antigenic determinants (development of epitopes). Thus, cell density and/or cell viability and/or cell morphology are good indicators of the success or otherwise of the cell culture parameters used. Adjustment of the parameters can then be made if unacceptable or sub-optimal cell density and/or cell viability is observed.

Whilst it is known to monitor these cell conditions, conventionally, their monitoring involves taking a sample from the cell culture and examining the sample under a microscope at the laboratory workbench, for example as described in U.S. Pat. No. 73,696,969, or using colorimetric, fluorometric or spectroscopic analysis of samples. Each time a sample is taken, there is a risk of contaminating the cell culture. It is vitally important in immunotherapy that no, or few, pathogens are administered to a patient, and so reducing the risk of contamination during cell culture is very important. More frequent monitoring with fewer steps, and automatic corrective alterations in the cell culture inputs provide a better chance of producing effective therapeutic cell cultures, in less time than would be possible with conventional multi-condition determination. This is of particular import for sensitive stem cell culturing.

Further, the inventors have realised that customers can be reluctant to spend significant amounts on disposal bioprocessing equipment so it is necessary to reduce costs where possible, which means that a low cost monitoring device is desirable, with the more expensive features of products being re-useable.

The problems mentioned above are addressed by embodiments of the invention described and illustrated herein, which fall within the ambit of the claims herein.

### SUMMARY OF THE INVENTION

Aspects of the invention are set out in the independent claims herein, with preferred features set out in dependent claims.

More advantages and benefits of the present invention will become readily apparent to persons skilled in the art in view of the detailed description below. Whilst the technical features of the invention are set out in the claims, the invention may extend to any combination of features described herein, not necessarily those features claimed. Further, the invention may be defined by only a portion of the features in any one claim combined with the whole, or a portion of the features from one or more other claims.

### DRAWINGS

The invention will now be described in more detail with reference to the appended drawings, wherein:

FIG. 1 shows schematically cell culture apparatus employing an in-process cell culture monitoring device;

FIG. 2 shows a sectional view of a cell monitoring device according to the invention;

FIGS. 3 and 3a show perspective and sectional views of part of the monitoring device similar to that shown in FIG. 2;

FIGS. 4, 5, and 6 each show sectional plan views of alternatives to the part shown in FIG. 3; and

FIG. 7 shows an image of cell, captured by a monitoring device.

### DETAILED DESCRIPTION

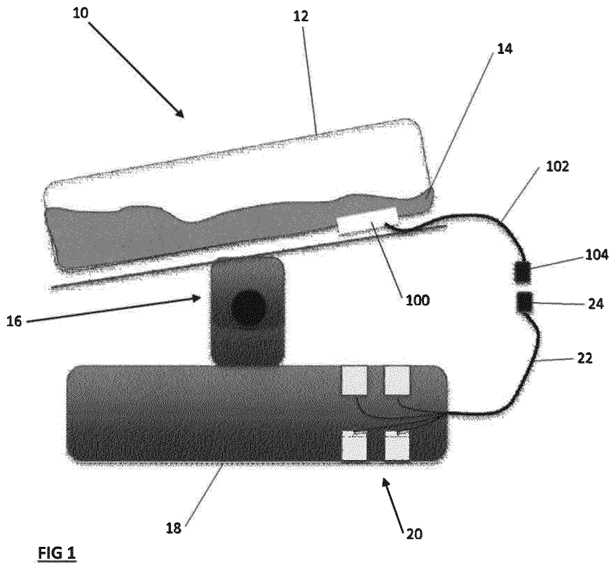

FIG. 1 shows cell culture apparatus 10, in this case comprising: a cell culture vessel 12, in this embodiment formed from a flexible bag, a cell culture mix 14, which is, depending on the length of cell culture, a mixture of mammalian cells which have a tendency to clump together, and liquid cell culture media, including known cell growth media. The apparatus 10 further includes a rocking platform 16 which gently mixes the cell mixture 14 while rocking. The platform 16 is supported on a base 18.

The cell culture mix 14 is monitorable by the monitoring device 100, which in this case is attached to the outside of the bag 12, and has an inlet and outlet, described in more detail below, in fluid communication with the interior of the bag 12 and, consequently, the cell culture mix 14. The device 100 further includes an umbilical 102 which includes, in this embodiment an electrical power supply, and signal lines (electrical and/or optical fibre).

The base 18 includes a complementary a microcontroller and associated hardware 20 for controlling the electrical supply to the device, as well as a data memory for buffering data coming from the device 100, all connected to the device via a complementary umbilical 22, and a connector 24 which mates with a complementary connector 104 on the end of the device's umbilical 102. In summary, the microcontroller is used to control the functioning of the device, and to receive data from the device. Whilst the microcontroller and its associated hardware 20 are preferably located in the base 18, they may be located elsewhere. The complementary connectors 24/104 are used for ease of assembly.

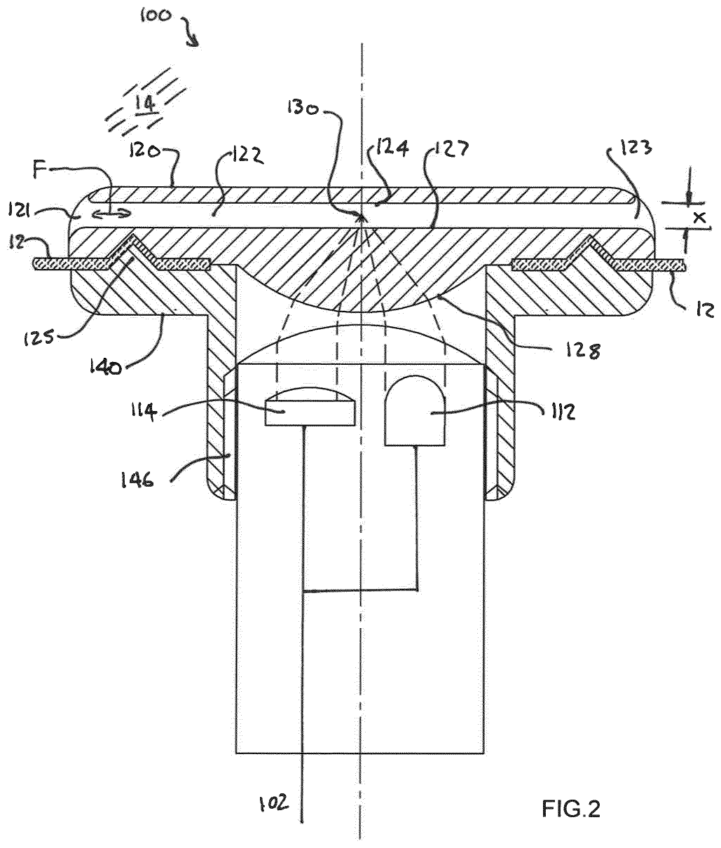

FIG. 2 shows the monitoring device 100 in more detail in cross section. The monitoring device 100 includes a base 140 and a cap 120. The base 140 supports a small microscope 110 which in turn provides images of the contents of a monitoring channel 122 formed in the cap 120. The base 140 and the cap 120 include complimentary formations 125 which clamp around the flexible bag 12, providing a hermetic seal around an opening formed in the bag 12. A heat fusible seal 125 is preferred, but adhesive or a mechanical clamp can be employed alternatively or additionally.

In use, the flow channel 122 is exposed to the culture mix 14, such that fluids, including cells can flow in the direction of arrows F through the channel 122. The cap 120 is formed from a transparent or translucent material, for example a PMMA plastics, such as acrylic plastics, and includes a partially spherical portion 128 which faces the microscope 110, which in turn has the effect of focusing light at a central region 130 of the channel 122, such that light emitted from an LED 112 of the microscope 110, is focused at the focal point region 130, and light emitted from that focal point is captured at an image array 114, in the microscope 110.

The base 140 includes an internal thread 146, which accepts a complimentary thread 126 formed on an outer surface of the microscope 110. Thus, rotation of the microscope about an axis a relative to the base 140 can adjust the position of the focal point region 130.

The cap 120, which may be in two halves to make the formation of the channel 122 easier, and the base 140 are intended to form an assembly along with the flexible bag 12. Since the assembly is intended to be disposable, then a removable microscope 110 is preferred in order that the microscope can be reused. So, whilst complimentary threads 126 and 146 have been illustrated, other readily releasable fastening between the base 140 and the microscope 110 could be employed. For example a bayonet fitting or a simple push fit could be employed.

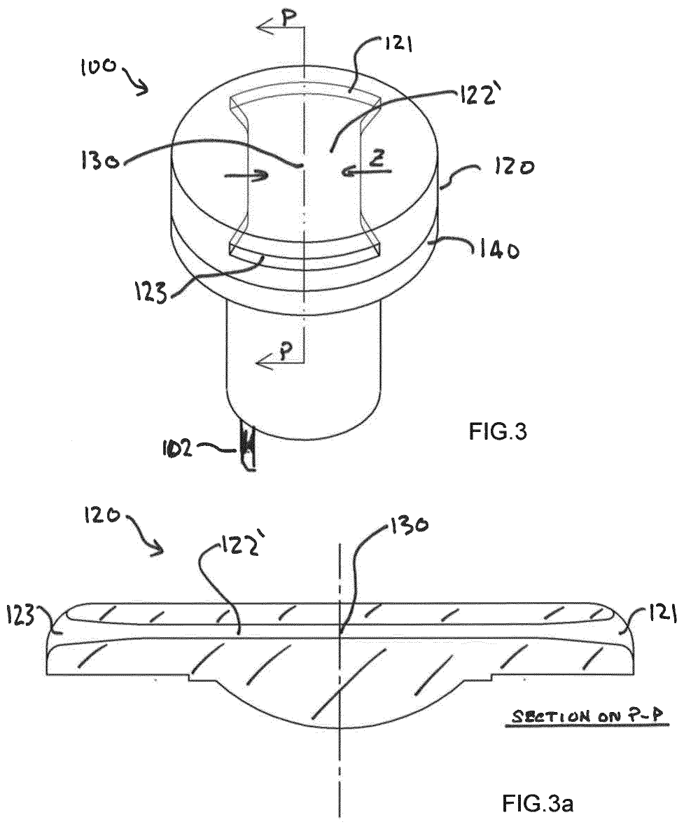

FIG. 3 shows an external pictorial view of the monitoring device 100. The cap 120 is, as described above, intended, at least at the domed spherical portion 128 to be light transmissive, and, since it is more convenient to mould the cap 120 as a unitary formation or in two halves, then, as shown in this illustration the whole cap is transparent. Consequently the channel 122′ is visible in this illustration. As can be seen in FIG. 2, the channel 122′ includes two openings 121 and 123 which function, one as an inlet and one as an outlet, depending on the direction of flow. The openings 121 and 123 are flared at their ends such that the mid-portion of the channel 122′ provides a restriction to the flow. In turn, this restriction chokes the flow and so reduces the flow rate at the focal point region 130, and improves the quality of the image obtained at that point, because the cells are moving more slowly.

FIG. 3a is a sectional view in the plane P-P visible in FIG. 3. In this view, it can be seen that channel 120′ is narrower at region 130, and differs from the more parallel channel 122 of FIG. 2. In this case the narrower channel acts funnel fluids through the channel, to produce a choked but longer monitored flow past the optical monitoring region 130.

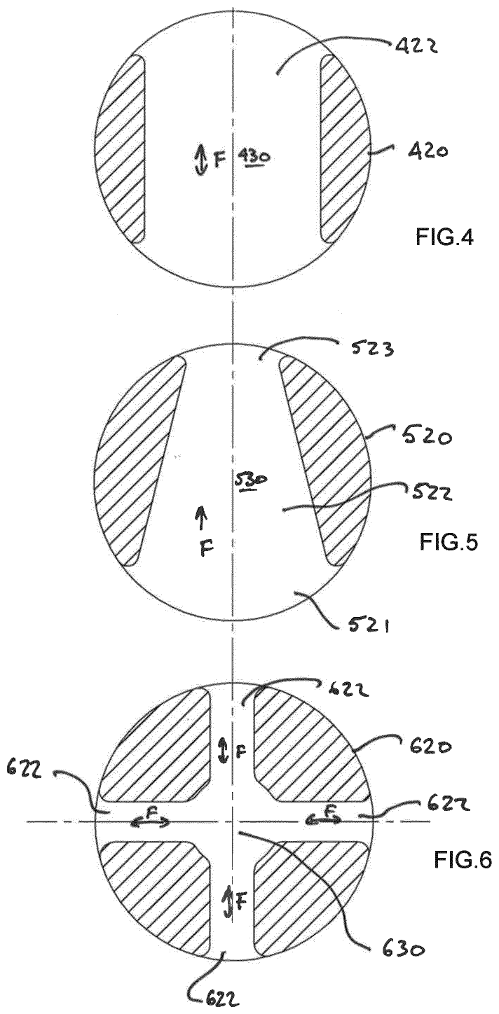

It will be understood that other flow path profiles could be employed. FIGS. 4, 5 and 6 each show sectional profiles of alternative flow parts in a plan view. In FIG. 4 an alternative cap 420 is illustrated, which includes a flow channel 422 somewhat wider than the flow channel 122 illustrated in FIG. 3. In this case, the depth X (see FIG. 3) of the flow channel 422 can be smaller then the depth of channel 122 in order to maintain a similar channel cross-sectional area to the channel cross-sectional area of the channel 122. Otherwise, the profile of the channel 422 is similar to that shown in FIG. 2. It can be seen that the cross-sectional area of the region 130 where the microscope will view the flow, is X (FIG. 2) multiplied by Z (FIG. 3), where Z is the width of the field of view of microscope, which is not necessarily the width of the channel. For easier calculations that cross-sectional area has been made constant for the caps 120 and 420.

An alternative cap 520 is shown in FIG. 5. In this case the channel 522 illustrated includes an inlet 521 and an outlet 523 it is apparent that the inlet has a larger cross-sectional area than the outlet, in order to restrict the flow of liquids in the direction of arrow F to enhance the quality of the image obtained.

It will be apparent that the caps shown in FIGS. 3 and 4 are bidirectional in the sense that flow can be imaged irrespective of the direction it flows through the caps. Further, it will be apparent that the cap 520 shown in FIG. 5 is intended for unidirectional flow in the direction of arrow F. A further alternative cap 620 is illustrated in FIG. 6. Here, the cap 620 provides for omnidirectional monitoring, in other words flow can enter and leave the cap irrespective of its approach direction. A central chamber 630 is used to provide cell images.

The cross sectional dimensions of the channels 122, 422, 522 and 622 do not need to be exact however, a depth (X) of 1 to 3 mm has been found to provide reasonably good results. In other words, cells move through such channels under the influence of gravity as the bioreactor bag 12 is rocked back and forth, and travel at a speed which allows them to be imaged. In all cases, the channels have generally parallel top and bottom walls with no regions where fluids can pool or come to a dead end. This means that the fluids in the monitor are constantly flushed and do not stagnate. This is particularly advantageous over existing cell sampling techniques which require drawing off of an aliquot of cell suspension often along a dead end which stagnates and causes a source of atrophy and subsequent contamination. It is important, but not essential to provide a uniform depth (X) of channel across the monitoring region 130 for consistent results. Where the flow becomes choked, for example as will happen in the channel 522 shown in FIG. 5, then constant flow speed can be assumed.

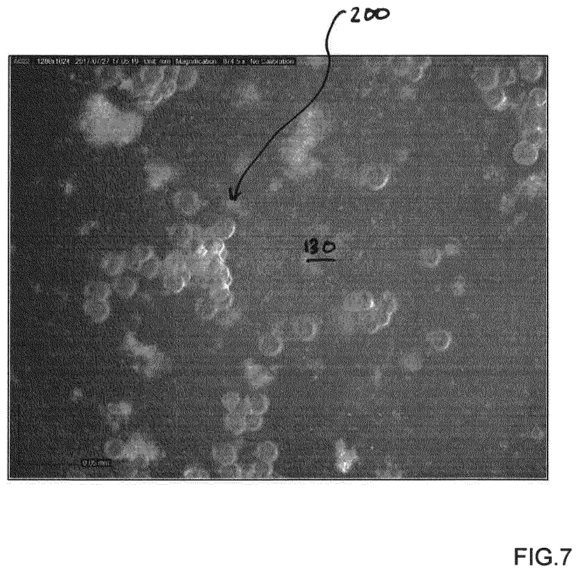

FIG. 7 shows a typical image obtained from the microscope 110 of a monitoring region 130, where, in this case, the lymphoblastoid cells 200 are visible. Successive images can be used to determine the velocity of the cells through the flow channel. However, since the channel has a relatively small cross-sectional area then the flow through the channel may be choked, resulting in a generally consistent flow rate which does not need to be determined continually. Image analysis software can determine also the number of cells in the image and if the cross-sectional area of the microscope viewing area and flow rate in the channel is known then the volume of fluid containing the cells and passing through the channel can be determined. Further, if the total volume of the cell culture 14 is known, and that can be determined for example by knowing the mass of the culture 14, in turn derived from a reading of a weighing scales built into the base 18, then an estimation of the total population of cells in the cell culture 14 can be made based on the number of cells passing through the channel for a given volume of fluid passing through the viewing region of the channel at the same time.

Image analysis software can be employed to count the number of cells newly appearing in an image, thereby providing a count of the cells over a period of time. The period of time can also be used to determine the total volume of fluids imaged because the cross-sectional area of flow at the image is also known.

Further, analysis of the images can determine the viability of the cells counted. For example, the inventors have observed that healthy cells have a well-defined cell wall such that an image captured the cells in the channel provides a clear contrast between the cell wall and the fluid suspension. Whereas, a dead or unhealthy cell provides a less distinct contrast. Image analysis of the contrast of the cell in relation to its background can give an indication of the viability of the cell in question. In practice, a light density value can be assigned to successive pixels from the array of the microscope image array 114, and if the rate of change of the density value of a cell wall area is sufficiently large, then that cell can be determined as healthy. Alternatively, where the rate of change of the density value is low then that is an indication that the cell wall is not healthy and that the cell is potentially not viable. Thus, with successive cell image analysis a reasonably accurate determination of cell viability can be obtained, for example as a percentage of the number of cells counted according to the method mentioned above.

In operation, the cell counting and assessment of cell viability can be performed by computer connected to the microcontroller 20. Such monitoring can be performed continually, periodically, or at random, during the cell culture process, and can be used to influence the parameters of the cell culture, or to determine when the cell density is of sufficient magnitude to end cell culture. This is particularly useful in autologous cell therapies where the characteristics of the seed cells are unknown. Since the channels are simple flat channels with no pockets of fluid that cannot drain away when tilted, then no dead pockets or pools can form, which means that the monitor is self draining and not a source of atrophy or contamination. Thus, it can be seen that the monitoring channel should have sufficient size and shape to allow self-draining of the channel after it is agitated or tilted, although in practice a residue of fluid may remain. However such residue will ultimately be washed away, for example after a low number of cycles, i.e no more than 9 or 10 cycles.

The monitoring and determination according to the above technique can be repeated multiple times, the cell culture process being monitored by the device and its determinations used to feedback to the controller which can adjust the parameters of the cell culture, for example increasing or decreasing nutrient supply or perfusion rates and determining when the viable cell density has reach the desired levels. Once cell culture is complete, the cell bag 12 and attached device can be discarded, but the microscope can be released and reused with the next bag 12.

The invention is not to be seen as limited by the embodiments described above, but can be varied within the scope of the appended claims as is readily apparent to the person skilled in the art. For instance, it is preferred that the cap 120 is positioned at the bottom of the cell culture 14, because the cells tend to congregate at the bottom of the suspension fluid. Further, many cell cultures start with a small volume of suspension fluid and increase the fluid volume as culture progresses. Thus, conveniently the cap is positioned at the bottom of the cell culture 14. However, provided that a flow of cells can be made to pass through the channel, then the cap can be positioned anywhere to provide reasonable results. So, in an alternative embodiment, the cap 120 or a similar arrangement including a flow channel can be provided on the end of a probe, which probe can be repositioned within a bioreactor. Such a probe can be inserted into a cell culture vessel, with the microscope preferably remaining outside the vessel. So long as a representative sample of cells is monitored the monitoring channel of the end of the probe which would include a monitoring channel as described above can be positioned anywhere in the vessel.

In the embodiment illustrated in FIG. 2, illuminating light is provided by an LED 112 positioned in the microscope 110, and supplied with power via the umbilical 102. Thus, the microscope, including the LED light can be reusable whereas the lower cost plastic parts (cap 120 base 140) are disposable along with the disposable bioreactor bag 12. However, with sufficient illumination the microscope 110 can function without its own light source. In that case, ambient illumination may be sufficient, or light internally reflected in the cap 112, for example provided by an LED formed in the base 140 could be employed. To enhance the illumination light, the far surface 124 of the channel 122 (or any far surfaces of the other channels illustrated) may be reflective, for example mirrored to provide more light to the microscope 110.

1. An in-process cell monitoring device comprising:
a monitoring flow channel having at least one inlet and at least one outlet exposeable to a cell culture;
a microscope positionable to view the contents of a region of the channel; and
a computer operable at least to count any cells in the region.

2. The device as claimed in claim 1, wherein the flow channel is illuminated.

3. The device as claimed in claim 1, wherein the flow channel, at least at said region, is transparent or translucent, and is a closed channel other than said inlet and outlet.

4. The device as claimed in claim 1, wherein the flow channel is self draining and optionally includes no pools or dead ends or areas of fluid stagnation.

5. The device as claimed in claim 1, wherein the flow channel at the region has a depth, in the direction of viewing, of about 1 mm to about 3 mm, which is a uniform depth over the viewing region area.

6. The device as claimed in claim 1, wherein the flow channel has a substantially uniform cross sectional area, or has a uniformly tapering cross sectional area at the region and optionally is increased in cross section area at the inlet and optionally at the outlet also.

7. The device as claimed in claim 1, wherein said at least one inlet and at least one outlet comprises two inlets and two outlets.

8. A bioreactor including a cell culture volume, and an in-process cell monitoring device, said device comprising:
a flow channel having at least one inlet and at least one outlet each in fluid communication with the volume, of sufficient cross-sectional area to allow fluids to drain from the inlet to the outlet; and
a microscope positionable to view the contents of a region of the channel.

9. The bioreactor as claimed in claim 8, wherein at least a majority of said channel is disposed within the culture volume, and said microscope is releasably held to the remaining device externally of said volume.

10. The cell culture apparatus including a bioreactor as claimed in claim 8, said apparatus further including a bioreactor mover, moveable with sufficient magnitude as to cause a portion of any fluid in the bioreactor to flow through the flow channel.

11. A method for monitoring a cell culture including determining cell density comprising, in any suitable order, the following steps:
a) causing a flow of fluid through a region of a monitoring channel of predetermined cross-sectional area;
b) capturing an image of the region;
c) using a computer, determining the number of cells imaged for a predetermined time;
d) using data from more than one image, determining the flow speed through measuring channel;
e) using the determined flow speed and cross-sectional area at the region, determining the volumetric flow rate in the channel; and
f) determining the cell density from said number of cells imaged and the volumetric flow rate.

12. The method of claim 11, wherein the step of determining the speed of flow includes analysis of successive captured cell images at known time intervals and/or the step of determining the volumetric flow rate includes multiplying the speed of flow by the cross-sectional area.

13. The method of claim 11, including the further step of determining the viability of the cells imaged, by means of assigning a light density value to successive pixels from a captured image, and determining the rate of change of the light density value of a cell wall area.

14. The method of claim 11, wherein the flow of fluid in step a) is the result is agitating a bioreactor vessel, for example tilting of the said bioreactor vessel.

15. The method of claim 14, wherein said fluid in said flow is substantially drained or exchanged with other fluid after each cycle of agitation, for example after each tilt or after a single figure number of tilts.
